(19) United States
(12) Patent Application Publication
(10) Pub. No.: US 2020/0397381 A1
(43) Pub. Date: Dec. 24, 2020

(54) INFORMATION PROCESSING DEVICE AND NON-TRANSITORY COMPUTER READABLE MEDIUM

(71) Applicant: FUJI XEROX CO., LTD., Tokyo (JP)

(72) Inventors: Tadashi SUTO, Kanagawa (JP); Tsutomu KIMURA, Kanagawa (JP); Kosuke AOKI, Kanagawa (JP)

(73) Assignee: FUJI XEROX CO., LTD., Tokyo (JP)

(21) Appl. No.: 16/726,934

(22) Filed: Dec. 26, 2019

(30) Foreign Application Priority Data
Jun. 18, 2019 (JP) 2019-112561

(51) Int. Cl. A61B 5/00; G06T 11/20; G06T 7/20; A61B 5/11; A61B 5/0205

(52) U.S. Cl. CPC A61B 5/721; G06T 11/206; G06T 7/20; A61B 5/1123; A61B 5/1126; A61B 5/053; A61B 5/0205; A61B 5/7425; A61B 5/6803; G06T 2207/30201; A61B 5/4205

(57) ABSTRACT

An information processing device includes an extractor and a controller. The extractor extracts biological information and a motion of a head from a potential measurement result, which is a result of measuring a potential in the head of a human body. The controller performs control to enable the biological information and the motion of the head that have been extracted by the extractor to be simultaneously presented in association with each other.

### CROSS-REFERENCE TO RELATED APPLICATIONS

This application is based on and claims priority under 35 USC 119 from Japanese Patent Application No. 2019-112561 filed Jun. 18, 2019.

### BACKGROUND

### (i) Technical Field

The present disclosure relates to an information processing device and a non-transitory computer readable medium.

### (ii) Related Art

Japanese Unexamined Patent Application Publication No. 2001-008915 discloses the following technique for providing a brain wave data obtaining device capable of measuring brain waves up to a high frequency range in rapid eye movement (REM) sleep. That is, a signal from an electrode or sensor for collecting an electroencephalogram (EEG), an electromyogram (EMG), and an electroculogram (EOG) is converted into digital data by an A/D converter through an amplifier at a sampling rate determined by a sampling controller and is stored in a waveform memory. These operations are controlled by an information processor. The waveform data stored in the waveform memory is printed as waveform data by a recording unit through an interface unit, for example. The sampling controller increases the sampling rate when the data of the EMG becomes flat and REM sleep is detected.

### SUMMARY

In a configuration including a sensor for measuring biological information and another sensor for measuring a motion of a head, measurement results of the sensors are obtained separately. Thus, even when there is a possibility that the biological information and the motion of the head are associated with each other, the measurement results are displayed separately.

Aspects of non-limiting embodiments of the present disclosure relate to an information processing device and a non-transitory computer readable medium that are capable of simultaneously presenting biological information and a motion of a head in a comparable manner, compared to a case where biological information and a motion of a head are measured by different sensors.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing device including an extractor and a controller. The extractor extracts biological information and a motion of a head from a potential measurement result, which is a result of measuring a potential in the head of a human body. The controller performs control to enable the biological information and the motion of the head that have been extracted by the extractor to be simultaneously presented in association with each other.

### BRIEF DESCRIPTION OF THE DRAWINGS

An exemplary embodiment of the present disclosure will be described in detail based on the following figures, wherein:

FIG. 1 is a conceptual module configuration diagram of an example configuration according to the exemplary embodiment;

FIG. 2A is an explanatory diagram illustrating an example system configuration utilizing the exemplary embodiment;

FIG. 2B is an explanatory diagram illustrating an example system configuration utilizing the exemplary embodiment;

FIG. 2C is an explanatory diagram illustrating an example system configuration utilizing the exemplary embodiment;

FIG. 3 is an explanatory diagram illustrating a specific example system configuration utilizing the exemplary embodiment;

FIG. 4 is a flowchart illustrating an example process according to the exemplary embodiment;

FIG. 5 is a flowchart illustrating an example process according to the exemplary embodiment;

FIG. 6 is a flowchart illustrating an example process according to the exemplary embodiment;

FIG. 7 is a flowchart illustrating an example process according to the exemplary embodiment;

FIG. 8 is an explanatory diagram illustrating an example process according to the exemplary embodiment;

FIG. 9 is an explanatory diagram illustrating an example process according to the exemplary embodiment;

FIG. 10 is an explanatory diagram illustrating an example process according to the exemplary embodiment;

FIGS. 11A to 11C are explanatory diagrams illustrating an example process according to the exemplary embodiment;

FIG. 12 is an explanatory diagram illustrating an example process according to the exemplary embodiment;

FIG. 13 is an explanatory diagram illustrating an example process according to the exemplary embodiment;

FIG. 14 is an explanatory diagram illustrating an example process according to the exemplary embodiment;

FIGS. 15A and 15B are explanatory diagrams illustrating an example process according to the exemplary embodiment;

FIG. 16 is an explanatory diagram illustrating an example process according to the exemplary embodiment;

FIGS. 17A and 17B are explanatory diagrams illustrating an example process according to the exemplary embodiment; and

FIG. 18 is a block diagram illustrating an example hardware configuration of a computer implementing the exemplary embodiment.

### DETAILED DESCRIPTION

Hereinafter, an exemplary embodiment for carrying out the present disclosure will be described with reference to the attached drawings.

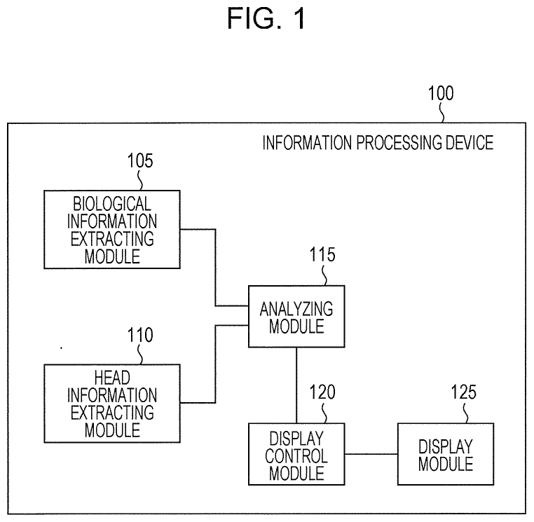

FIG. 1 is a conceptual module configuration diagram of an example configuration according to the exemplary embodiment.

Modules are components of software (including computer programs as the interpretation of “software”) or hardware that can be logically separated from one another in general. Thus, the modules according to the exemplary embodiment include not only modules in a computer program but also modules in a hardware configuration. Therefore, the description of the exemplary embodiment includes a description of a computer program for causing a computer to function as those modules (for example, a program for causing a computer to execute individual steps, a program for causing a computer to function as individual units, or a program for causing a computer to implement individual functions), a system, and a method. For the convenience of description, “store”, “cause . . . to store”, or an expression equivalent thereto may be used. These expressions mean “cause a storage device to store” or “perform control to cause a storage device to store” in a case where an exemplary embodiment is a computer program. The modules may correspond to functions on a one-to-one basis. In terms of packaging, a single module may be constituted by a single program, plural modules may be constituted by a single program, or a single module may be constituted by plural programs. Plural modules may be executed by a single computer, or a single module may be executed by plural computers in a distributed or parallel environment. Alternatively, a single module may include another module. Hereinafter, the term “connection” will be used to refer to a logical connection (for example, transmission and reception of data, instructions, a referential relationship between pieces of data, login, etc.) as well as a physical connection. The term “predetermined” means being determined before target processing, and includes the meaning of being determined in accordance with a present situation/state or a previous situation/state, before target processing before or after processing according to the exemplary embodiment starts. In a case where there are plural “predetermined values”, the plural predetermined values may be different from one another, or two or more of the values (of course including all the values) may be the same. A description “in the case of A, B is performed” is used as the meaning “whether A or not is determined, and B is performed if it is determined A”, except for a case where the determination of whether A or not is unnecessary. Enumeration of items, such as “A, B, and C”, is merely enumeration of examples unless otherwise noted, and includes selection of only one of them (for example, only A).

A system or device may be constituted by plural computers, hardware units, devices, or the like connected to one another through a communication medium, such as a network (“network” includes communication connections on a one-to-one basis), or may be constituted by a single computer, hardware unit, device, or the like. The terms “device” and “system” are used synonymously. Of course, “system” does not include a man-made social “organization” (i.e., a social system).

Target information is read from a storage device in individual processing operations performed by respective modules or in individual processing operations when plural processing operations are performed within a module. After each processing operation is performed, a result of the processing is written into the storage device. Thus, a description of reading from the storage device before a processing operation and writing into the storage device after a processing operation may be omitted. Examples of the storage device include a hard disk drive, a random access memory (RAM), an external storage medium, a storage device connected through a communication line, a register in a central processing unit (CPU), and the like.

An information processing device 100 according to the exemplary embodiment has a function of simultaneously presenting biological information and a motion of a head in association with each other and includes, as illustrated in the example in FIG. 1, a biological information extracting module 105, a head information extracting module 110, an analyzing module 115, a display control module 120, and a display module 125.

The “biological information” herein means information obtained by measuring a vital activity of a human body. Examples of the biological information include information about an electrocardiogram, heart rate, blood pressure, body temperature, brain wave, myoelectric potential, and retinal (fundus) potential. In the exemplary embodiment, brain wave information is mainly used as an example.

The “head” herein means a portion including a neck and thereabove and includes, for example, any one or more of ears, mouth, throat, eyes, nose, forehead, cheeks, and the like.

The “motion of the head” herein means a motion of the whole or part of the head and includes, for example, nodding, head shaking, chewing, swallowing, winking, breathing, a motion of corners of the mouth, and the like. Specifically, a motion of corners of the mouth may be determined by using myoelectric potential information and a face image. For this purpose, measurement of biological information and capturing of a face image may be simultaneously performed, and potential data and a face image may be displayed and presented in association with each other.

The biological information extracting module 105 is connected to the analyzing module 115. The biological information extracting module 105 extracts biological information from a potential measurement result, which is a result of measuring a potential in the head of a human body.

In addition, the biological information extracting module 105 may extract waves in plural frequency bands from the potential measurement result.

Here, examples of a “wave in a frequency band” include an α (alpha) wave, a β (beta) wave, a γ (gamma) wave, a θ (theta) wave, and a δ (delta) wave. The “waves in plural frequency bands” mean waves in two or more of these frequency bands.

The biological information extracting module 105 may perform fast Fourier transform (FFT) on the potential measurement result to extract brain waves.

The head information extracting module 110 is connected to the analyzing module 115. The head information extracting module 110 extracts a motion of the head.

In addition, the head information extracting module 110 may extract a motion of the head from a potential measurement result, which is a result of measuring a potential in the head of a human body.

The analyzing module 115 is connected to the biological information extracting module 105, the head information extracting module 110, and the display control module 120. The analyzing module 115 analyzes the relationship between brain wave information and a motion of the head by using brain wave information, which is the biological information extracted by the biological information extracting module 105, and the motion of the head extracted by the head information extracting module 110.

In addition, the analyzing module 115 may analyze an action of a user.

The display control module 120 is connected to the analyzing module 115 and the display module 125. The display control module 120 performs control to enable the biological information extracted by the biological information extracting module 105 and the motion of the head extracted by the head information extracting module 110 to be simultaneously presented in association with each other.

In addition, the display control module 120 may perform control to display, on the screen of the display module 125 in a comparable manner, plural waves extracted by the biological information extracting module 105 and the motion of the head.

The display module 125 is connected to the display control module 120. The display module 125 performs display on the screen, such as a liquid crystal display or an organic electroluminescence (EL) display, in accordance with the control of the display control module 120.

For example, the motion of the head may include chewing.

In this case, the biological information extracting module 105 may extract, as a chewing portion, a portion that matches a predetermined pattern in the potential measurement result.

The “predetermined pattern” herein may be that a first peak of a graph is higher than a predetermined first threshold value or is higher than or equal to the first threshold value and that a second peak after the first peak is lower than the first threshold value or is lower than or equal to the first threshold value and is higher than a predetermined second threshold value or is higher than or equal to the second threshold value, the second threshold value being smaller than the first threshold value.

The display control module 120 may perform control to display in a comparable manner the biological information and the chewing portion that is on the graph indicating the potential measurement result.

For example, the motion of the head may include swallowing.

In this case, the analyzing module 115 may analyze that a portion that matches a predetermined pattern in the potential measurement result is a swallowing portion.

The “predetermined pattern” herein may be that a third peak of the graph after chewing is higher than a predetermined third threshold value or is higher than or equal to the third threshold value.

The display control module 120 may perform control to display in a comparable manner the biological information and the swallowing portion that is on the graph indicating the potential measurement result.

In this case, if the analyzing module 115 analyzes that the motion of the head is a predetermined motion by using a measurement result of an acceleration sensor put on the head, the analyzing module 115 may extract swallowing at a portion that matches a predetermined pattern in the potential measurement result as swallowing of drink.

The “predetermined motion” herein may be a motion of tilting the head backward.

The display control module 120 may perform control to display information indicating swallowing of drink.

The display control module 120 may perform control to simultaneously display results of plural types of transform processes on the potential measurement result. For example, an obtained potential measurement result may be processed by plural types of transform processes, such as FFT, wavelet transform, Stockwell transform, and empirical mode decomposition (a function arbitrarily set by a user), and the results of the transform processes may be simultaneously displayed.

In addition, the display control module 120 may perform control to display a graph showing an intensity ratio as a result of frequency analysis, or to display an overall intensity ratio as a result of frequency analysis.

The display control module 120 may perform control to display results of spectrum analysis for a predetermined period using a low-pass filter (LPF) and a high-pass filter (HPF) and to display other data that does not have periodicity.

The display control module 120 may perform control to display individual graphs showing results of transform processes such as FFT in a superimposed manner, may allow a user to select any of the graphs, and may perform control to display the graph selected by the user in an emphasized manner. Emphasized display includes, for example, changing the color or shape of the graph, and highlighted display.

The display control module 120 may allow a mode of a vertical-axis scale of the graph to be selected from among a mode of adjusting the scale in accordance with a maximum value or/and a minimum value, a mode of arbitrarily fixing a value display range in accordance with a user operation, and a mode of actively fixing the scale with a maximum value or/and a minimum value.

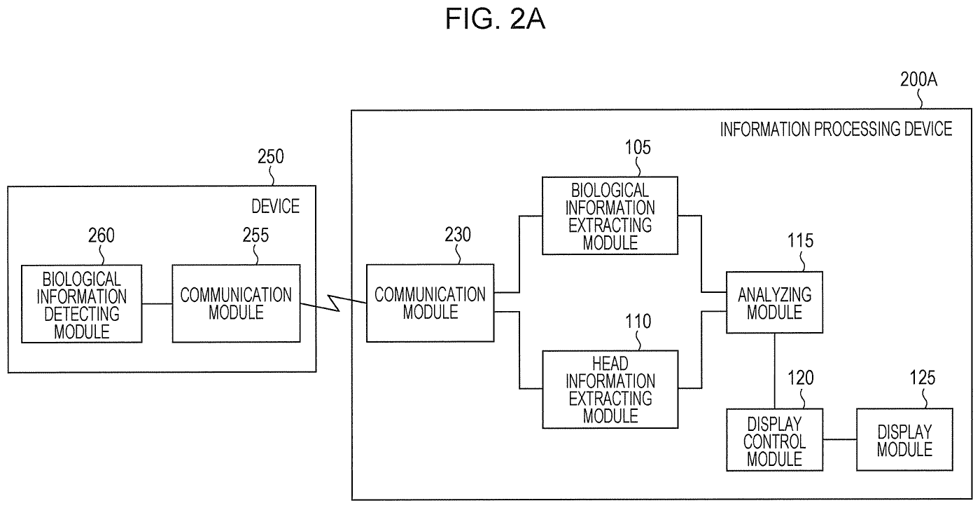

FIG. 2A is an explanatory diagram illustrating an example system configuration utilizing the exemplary embodiment. In this example system configuration, an information processing device 200A and a device 250 communicate with each other. The information processing device 200A has the configuration of the information processing device 100. Components of the same types as those illustrated in the above-described figure are denoted by the same reference numerals, and duplicate description will be omitted (the same applies hereinafter).

The device 250 includes a communication module 255 and a biological information detecting module 260.

The communication module 255 is connected to the biological information detecting module 260 and is connected to a communication module 230 of the information processing device 200A through a communication line. The communication module 255 communicates with the information processing device 200A. The communication line may be a wireless link, a wired link, or a combination thereof. For example, near field wireless communication, such as Wi-Fi or Bluetooth (registered trademark), may be used as a wireless link.

The biological information detecting module 260 is connected to the communication module 255. The device 250 is attached to, for example, the head of a user. The device 250 measures a potential in the head of the user wearing the device 250. For example, the electrodes described in Japanese Unexamined Patent Application Publication No. 2019-024758 (the electrodes that are made of a foam material, that have conductivity at least in a portion touching a living body, and that detect a brain wave while being in contact with a living body) may be used.

The biological information detecting module 260 transmits a potential measurement result to the communication module 255, and the communication module 255 transmits the potential measurement result to the information processing device 200A.

The information processing device 200A includes the biological information extracting module 105, the head information extracting module 110, the analyzing module 115, the display control module 120, the display module 125, and the communication module 230.

The communication module 230 is connected to the biological information extracting module 105 and the head information extracting module 110, and is connected to the communication module 255 of the device 250 through the communication line. The communication module 230 communicates with the device 250 to receive a potential measurement result. The communication module 230 transmits the potential measurement result received from the device 250 to the biological information extracting module 105 and the head information extracting module 110.

The biological information extracting module 105 is connected to the analyzing module 115 and the communication module 230. The biological information extracting module 105 extracts biological information from the potential measurement result, which is a result of measuring a potential in the head of a human body, received from the device 250.

The head information extracting module 110 is connected to the analyzing module 115 and the communication module 230. The head information extracting module 110 extracts a motion of the head from the potential measurement result, which is a result of measuring a potential in the head of a human body, received from the device 250.

FIG. 2B is an explanatory diagram illustrating an example system configuration utilizing the exemplary embodiment. In this example system configuration, an information processing device 200B and the device 250 communicate with each other. The information processing device 200B has the configuration of the information processing device 100.

The information processing device 200B includes the biological information extracting module 105, the head information extracting module 110, the analyzing module 115, the display control module 120, the display module 125, and the communication module 230.

The communication module 230 is connected to the biological information extracting module 105, and is connected to the communication module 255 of the device 250 through the communication line. The communication module 230 communicates with the device 250 to receive a potential measurement result. The communication module 230 transmits the potential measurement result received from the device 250 to the biological information extracting module 105.

The biological information extracting module 105 is connected to the analyzing module 115 and the communication module 230. The biological information extracting module 105 extracts biological information from the potential measurement result, which is a result of measuring a potential in the head of a human body, received from the device 250.

The head information extracting module 110 includes an image capturing module 235 and is connected to the analyzing module 115. The image capturing module 235 captures an image of the head of a user carrying the information processing device 200B (this user is the same as the user wearing the device 250). The image to be captured may be a still image or a moving image. In the case of a still image, two or more still images may be captured at different times according to one exemplary embodiment.

The head information extracting module 110 extracts a motion of the head from the image of the head of the user captured by the image capturing module 235. For example, the head information extracting module 110 may extract a motion of the head from the image by using a learning model generated through machine learning.

The display control module 120 performs control to display the image captured by the image capturing module 235. At this time, a graph indicating the biological information and the motion of the head may be displayed together with the image captured by the image capturing module 235. In addition, an image captured when the head moves may be displayed together.

FIG. 2C is an explanatory diagram illustrating an example system configuration utilizing the exemplary embodiment. In this example system configuration, an information processing device 200C and the device 250 communicate with each other. The information processing device 200C has the configuration of the information processing device 100.

The information processing device 200C includes the biological information extracting module 105, the head information extracting module 110, the analyzing module 115, the display control module 120, the display module 125, and the communication module 230.

The communication module 230 is connected to the biological information extracting module 105 and the head information extracting module 110, and is connected to the communication module 255 of the device 250 through the communication line. The communication module 230 communicates with the device 250 to receive a potential measurement result. The communication module 230 transmits the potential measurement result received from the device 250 to the biological information extracting module 105 and the head information extracting module 110.

The biological information extracting module 105 is connected to the analyzing module 115 and the communication module 230. The biological information extracting module 105 extracts biological information from the potential measurement result, which is a result of measuring a potential in the head of a human body, received from the device 250.

The head information extracting module 110 includes the image capturing module 235 and is connected to the analyzing module 115 and the communication module 230. The head information extracting module 110 extracts a motion of the head from the potential measurement result received from the device 250 and the image of the head of the user captured by the image capturing module 235. The head information extracting module 110 has both the function of the head information extracting module 110 illustrated in the example in FIG. 2A and the function of the head information extracting module 110 illustrated in the example in FIG. 2B. For example, when the motion of the head extracted from the potential measurement result received from the device 250 matches the motion of the head extracted from the image of the head, the head information extracting module 110 may extract a motion of the head. When both do not match, the head information extracting module 110 may determine not to extract a motion of the head or may adopt any one of the results as a motion of the head.

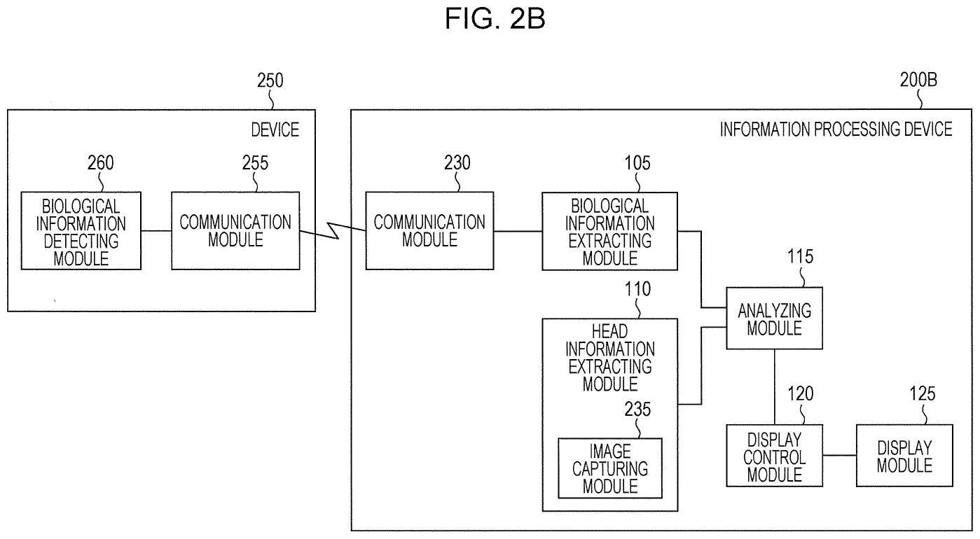

FIG. 3 is an explanatory diagram illustrating a specific example system configuration utilizing the exemplary embodiment.

A smartphone 300 is a specific example of the information processing device 200 (in particular, the information processing device 200B or the information processing device 200C), and a wearable device 350 is a specific example of the device 250. The smartphone 300 includes a camera 335 and captures an image of the head of a user 390. The camera 335 is a specific example of the image capturing module 235.

The user 390 carries the smartphone 300 and wears the wearable device 350 on the head. The smartphone 300 and the wearable device 350 communicate with each other by using near field wireless communication.

With use of the smartphone 300 and the wearable device 350, biological information and a motion of the head of the user 390 are displayed in association with each other on the screen of the smartphone 300. For example, chewing and swallowing of the user 390 are sensed.

As the wearable device 350, sensors are disposed in external auditory canals, and the wearable device 350 detects data of a composite waveform of a brain wave and a myoelectric potential (so-called raw data, hereinafter also referred to as raw data). The wearable device 350 transmits the raw data to the smartphone 300. The smartphone 300 regards the raw data as myoelectric potential data, and performs FFT on the raw data to convert the raw data to brain wave data. In other words, the smartphone 300 obtains two pieces of information, that is, brain wave data as biological information and myoelectric potential data indicating a motion of the head, from one piece of waveform data (raw data). For example, the smartphone 300 detects chewing and swallowing from the myoelectric potential data, marks a graph of the myoelectric potential data, and displays the graph of the myoelectric potential data and a graph of the brain wave data in the same time series. Accordingly, the user 390 is able to know the state of the brain wave data when chewing and swallowing are performed.

Specifically, the smartphone 300 extracts peaks on the graph of the myoelectric potential data, and compares the peaks with threshold values to detect chewing and swallowing. Subsequently, the smartphone 300 puts marks of chewing and swallowing on the graph of the myoelectric potential data. In addition, the smartphone 300 performs FFT analysis on the raw data, removes a noise frequency component, analyses a brain wave to generate a graph of the brain wave, and displays the graph of the brain wave and the graph of the myoelectric potential data in the same time series.

With use of the camera 335 of the smartphone 300, an image of the head, mainly the face, of the user 390 may be captured, and a motion of the face and a brain wave may be displayed in association with each other. A motion of the face corresponds to, for example, a facial exercise for beauty, training of facial muscles of expression, or the like.

In a case where the electrodes described in the above-mentioned Japanese Unexamined Patent Application Publication No. 2019-024758 is used for the sensors of the wearable device 350, the sensors serve as electrometers that measure a very low potential (for example, a potential of several μV) and are requested to obtain a signal of a high S/N ratio even in a slight change in potential.

However, when a slight change in potential can be obtained, a myoelectric potential generated from a muscle around the electrodes may be included as noise in a signal.

In the exemplary embodiment, a motion of the head is detected by using myoelectric potential information that is normally dealt with as noise. A motion of the head includes, for example, chewing, swallowing, and the like.

The wearable device 350, which performs measurement by using brain wave measuring electrodes disposed in external auditory canals and a ground (GND) disposed near an ear, is capable of obtaining a myoelectric potential signal from a motion of chewing, swallowing, or the like because there are large muscles of jaws, cheeks, and throat near the wearable device 350.

In the case of using the electrodes described in Japanese Unexamined Patent Application Publication No. 2019-024758, the following process is performed in the exemplary embodiment.

(1) The brain wave electrodes of the wearable device 350 are disposed in the external auditory canals of the user 390.

(2) Raw data is myoelectric potential data, an FFT process is performed on the raw data to obtain brain wave data, and two pieces of biological information are obtained from one piece of waveform data.

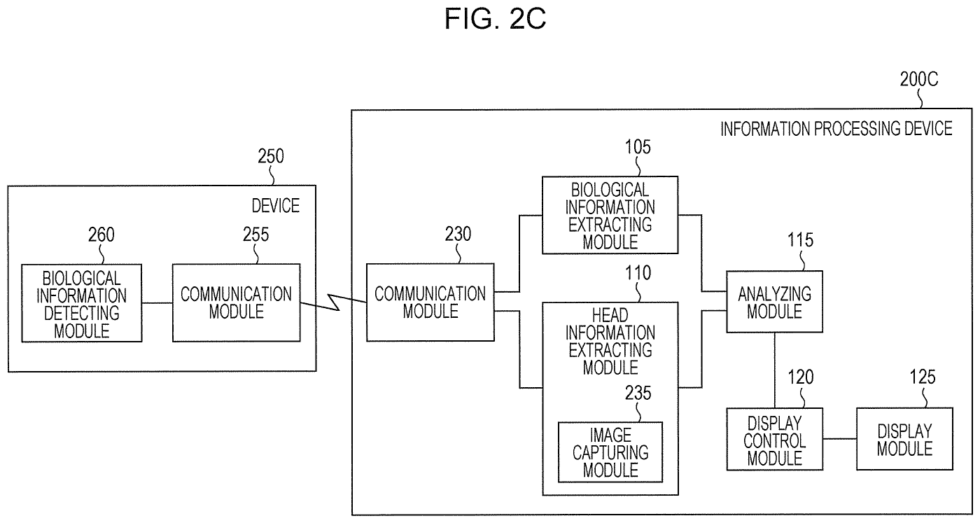

FIG. 4 is a flowchart illustrating an example process performed by the wearable device 350 according to the exemplary embodiment.

In step S402, the wearable device 350 detects biological information of the user 390. The biological information corresponds to the raw data described above.

In step S404, the wearable device 350 generates data to be transmitted to the smartphone 300.

In step S406, the wearable device 350 transmits the data to the smartphone 300. As described above, for example, the smartphone 300 and the wearable device 350 communicate with each other by using near field wireless communication.

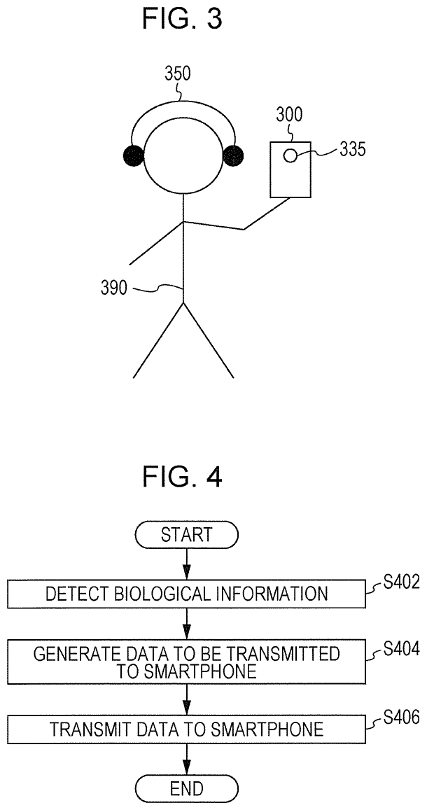

FIG. 5 is a flowchart illustrating an example process performed by the smartphone 300 according to the exemplary embodiment.

In step S502, the smartphone 300 receives data from the wearable device 350.

In step S504, the smartphone 300 extracts information about a brain wave from the received data.

In step S506, the smartphone 300 extracts information about a motion of the head from the received data.

In step S508, the smartphone 300 simultaneously displays both a graph of the information about the brain wave and a graph of the information about the motion of the head. Obviously, the two graphs have the same time series. Specifically, X axes indicating time of the two graphs may be caused to match each other, or the two graphs may be displayed in one region in a superimposed manner.

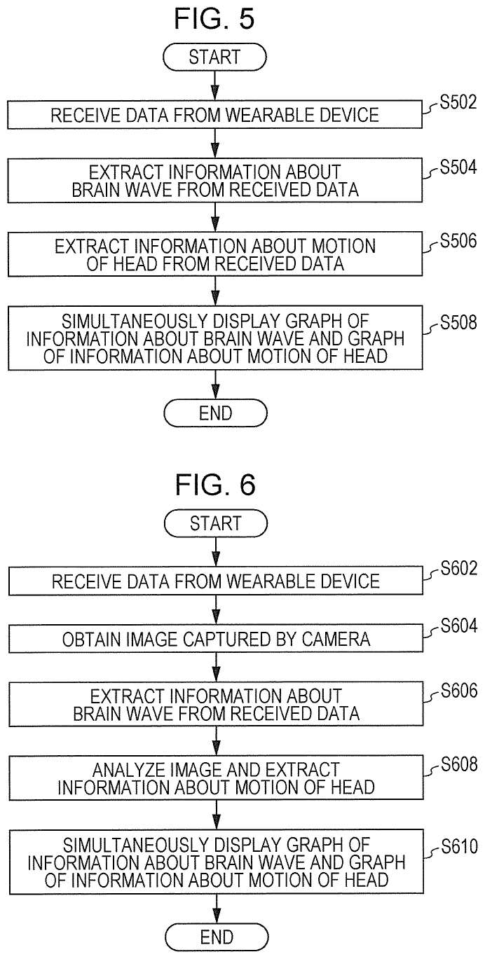

FIG. 6 is a flowchart illustrating an example process performed by the smartphone 300 according to the exemplary embodiment.

In step S602, the smartphone 300 receives data from the wearable device 350.

In step S604, the smartphone 300 obtains an image captured by the camera 335 of the smartphone 300.

In step S606, the smartphone 300 extracts information about a brain wave from the received data.

In step S608, the smartphone 300 analyses the image and extracts information about a motion of the head.

In step S610, the smartphone 300 simultaneously displays both a graph of the information about the brain wave and a graph of the information about the motion of the head. For example, a mark may be put on the time point where the motion of the head occurred on the graph of the brain wave.

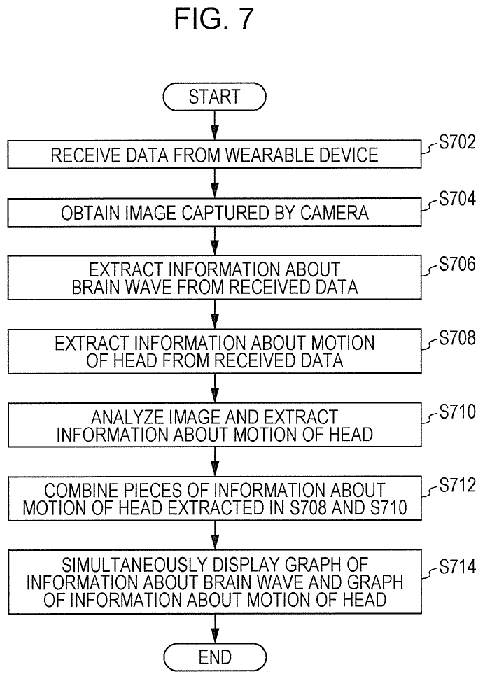

FIG. 7 is a flowchart illustrating an example process performed by the smartphone 300 according to the exemplary embodiment. This is a combination of the example process illustrated in the example in FIG. 5 and the example process illustrated in the example in FIG. 6.

In step S702, the smartphone 300 receives data from the wearable device 350.

In step S704, the smartphone 300 obtains an image captured by the camera 335.

In step S706, the smartphone 300 extracts information about a brain wave from the received data.

In step S708, the smartphone 300 extracts information about a motion of the head from the received data.

In step S710, the smartphone 300 analyses the image and extracts information about a motion of the head.

In step S712, the smartphone 300 combines the information about the motion of the head extracted in step S708 and the information about the motion of the head extracted in step S710.

In step S714, the smartphone 300 simultaneously displays both a graph of the information about the brain wave and a graph of the information about the motion of the head.

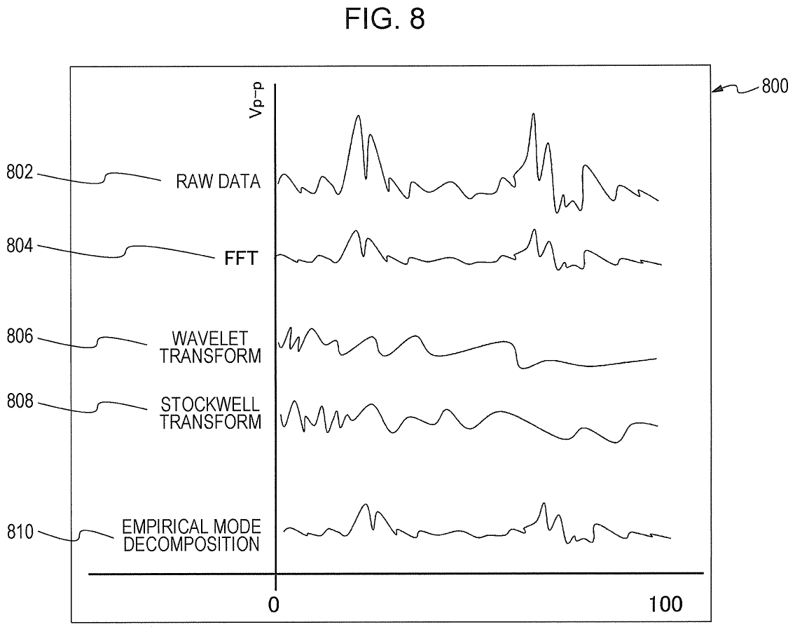

FIG. 8 is an explanatory diagram illustrating an example process according to the exemplary embodiment.

For example, a screen 800 of the smartphone 300 includes a raw data field 802, an FFT field 804, a wavelet transform field 806, a Stockwell transform field 808, and an empirical mode decomposition field 810.

A signal obtained from the wearable device 350 is subjected to a plurality of types of processes, such as FFT, wavelet transform, Stockwell transform, and empirical mode decomposition (a function arbitrarily set by a user), and results of the processes are displayed on the screen 800. Here, individual graphs are displayed in the raw data field 802, the FFT field 804, the wavelet transform field 806, the Stockwell transform field 808, and the empirical mode decomposition field 810, which have the same horizontal axis serving as a time axis.

An intensity ratio obtained as a result of frequency analysis may be displayed in the form of a graph, and an entire intensity ratio as a result of frequency analysis may be displayed.

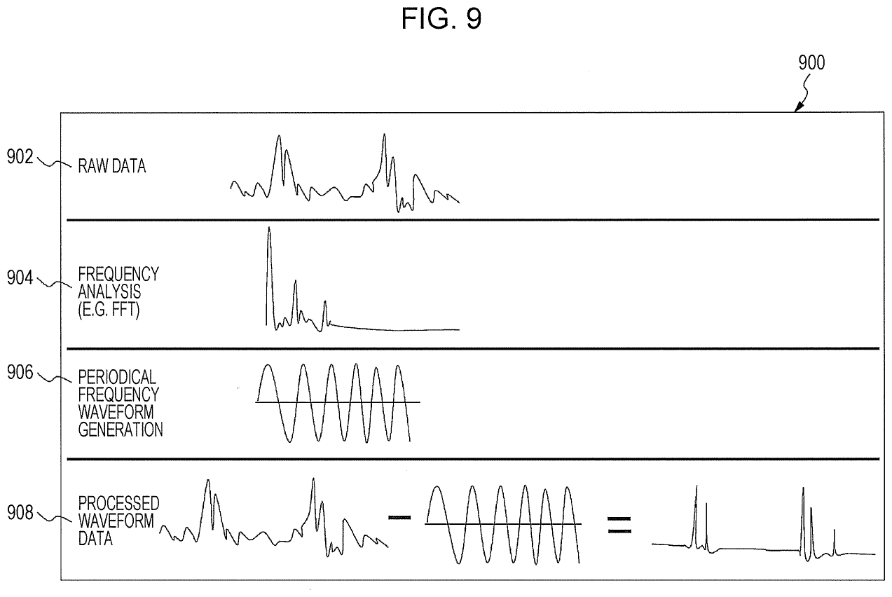

FIG. 9 is an explanatory diagram illustrating an example process according to the exemplary embodiment. In this example, raw data of biological information, data obtained by analyzing the biological information, and data obtained by processing the biological information (data obtained by removing a periodical component from the raw data) are displayed.

A screen 900 includes a raw data field 902, a frequency analysis field 904, a periodical frequency waveform generation field 906, and a processed waveform data field 908.

Periodical frequency components appear in the graph in the frequency analysis field 904. In FIG. 9, peaks in the graph in the frequency analysis field 904 correspond to the periodical frequency components.

In the processed waveform data field 908, for example, a graph generated from the graph in the raw data field 902 and the graph in the periodical frequency waveform generation field 906 is displayed. That is, as a result of removing periodical frequency components from the raw data, a waveform without periodicity of a myoelectric potential or the like can be seen easily.

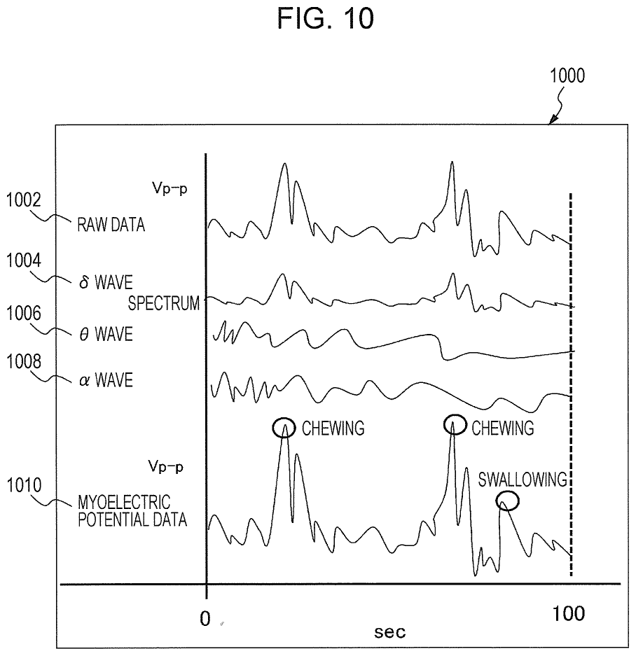

FIG. 10 is an explanatory diagram illustrating an example process according to the exemplary embodiment.

In this example, the portions of chewing and swallowing are marked on a graph.

A screen 1000 includes a raw data field 1002, a δ wave field 1004, a θ wave field 1006, an α wave field 1008, and a myoelectric potential data field 1010. The graphs in the δ wave field 1004, the θ wave field 1006, and the α wave field 1008 are obtained by performing a process such as FFT on raw data to divide the raw data into a δ wave, a θ wave, and an α wave. In the myoelectric potential data field 1010, a graph of myoelectric potential data extracted by removing a brain wave component from the raw data is displayed. On this graph, the portions of chewing and swallowing are marked.

Regarding chewing, if a first peak of the graph is higher than a predetermined first threshold value or is higher than or equal to the first threshold value, and if a second peak after the first peak is lower than the first threshold value or is lower than or equal to the first threshold value and is higher than a predetermined second threshold value or is higher than or equal to the second threshold value, the first peak is regarded as a chewing portion. The second threshold value is smaller than the first threshold value. In the example in FIG. 10, the portions of first peaks marked as chewing portions satisfy the condition that the first peaks are higher than the first threshold value and the second peaks following the first peaks are lower than the first threshold value and are higher than the second threshold value.

Regarding swallowing, if a third peak after a chewing portion is higher than a predetermined third threshold value or is higher than or equal to the third threshold value, the third peak is regarded as a swallowing portion. In the example in FIG. 10, the portion of a third peak marked as a swallowing portion satisfies the condition that the third peak is after a first peak and is higher than the third threshold value. Alternatively, a portion that is after a first peak and a second peak and that is higher than the third threshold value may be regarded as a swallowing portion.

With such display being performed, when chewing and swallowing are detected from myoelectric potential data, the relationship with individual brain waves may be checked on one screen, and thus hypothesizing or verifying may be easily performed.

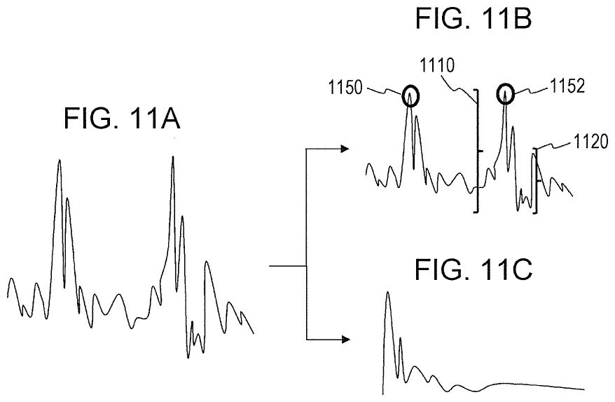

FIGS. 11A to 11C are explanatory diagrams illustrating an example process according to the exemplary embodiment.

The graph illustrated in the example in FIG. 11A is a graph of raw data. That is, brain wave data and myoelectric potential data are combined.

The raw data is divided into brain wave data and myoelectric potential data. For example, an FFT process may be performed on the raw data to generate brain wave data. The raw data may be dealt with as myoelectric potential data. That is, two pieces of information are obtained from one piece of raw data obtained from the wearable device 350.

The graph illustrated in the example in FIG. 11B is a graph of myoelectric potential data. Here, chewing portions 1150 and 1152 are higher than a threshold value 1110, and the peaks immediately thereafter are higher than a threshold value 1120. Thus, the chewing portions 1150 and 1152 are determined to be portions of chewing.

The graph illustrated in the example in FIG. 11C is a graph of brain wave data obtained by FFT analyzing the raw data and removing a noise frequency component.

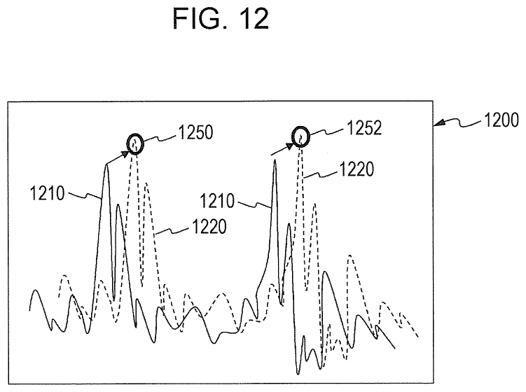

FIG. 12 is an explanatory diagram illustrating an example process according to the exemplary embodiment. The wearable device 350 may include an acceleration sensor in addition to a myoelectric potential sensor. In this case, information may be displayed also by using a detection result of the acceleration sensor.

The data of an acceleration data graph 1210 is obtained by the acceleration sensor in the wearable device 350 put on the head of the user 390. As the acceleration sensor, for example, a six-axis sensor or the like may be used.

The data of a raw data graph 1220 is obtained by the myoelectric potential sensor in the wearable device 350.

As a result of associating a motion of the head with information about a myoelectric potential of jaws by using a detection result of the acceleration sensor in the wearable device 350, the motion can be classified more specifically. For example, when information indicating swallowing with the head tilted backward is obtained, the motion is determined to be a motion of drinking.

In the example in FIG. 12, there are peaks in the acceleration data graph 1210 immediately before chewing portions 1250 and 1252 in the raw data graph 1220, and thus it is understood that there is a motion of tilting the head backward immediately before swallowing. This graph indicates acceleration in a front-back direction. Use of acceleration in a lateral direction makes it possible to determine tilting of a face or the like.

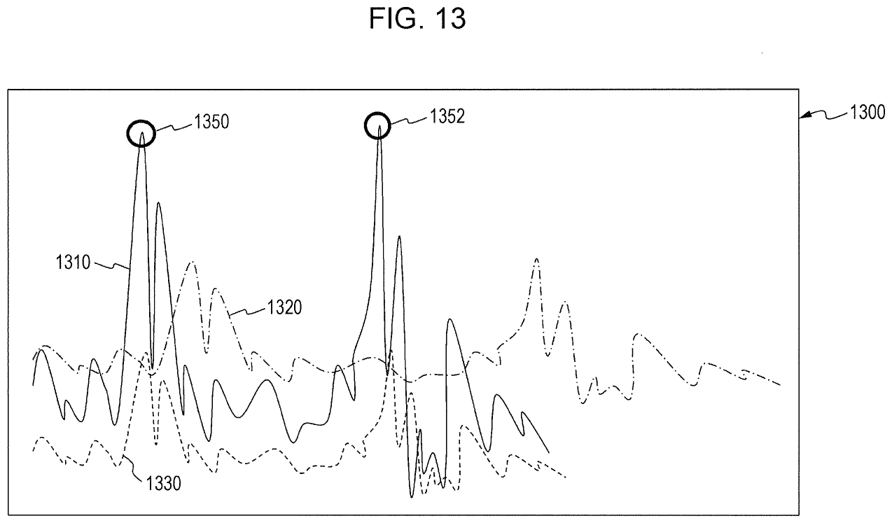

FIG. 13 is an explanatory diagram illustrating an example process according to the exemplary embodiment.

A raw data graph 1310, a brain wave (α wave) graph 1320, and a brain wave (β wave) graph 1330 are displayed on a screen 1300 in a superimposed manner. Obviously, the same horizontal axis serving as a time axis is used for these graphs.

In this example, the user is allowed to select a graph and highlight the graph. For example, when the brain wave (α wave) graph 1320 is selected by the user, the brain wave (α wave) graph 1320 may be displayed in red.

With the portions of chewing such as chewing portions 1350 and 1352 being marked, the state of the brain wave while the chewing is being performed can be observed.

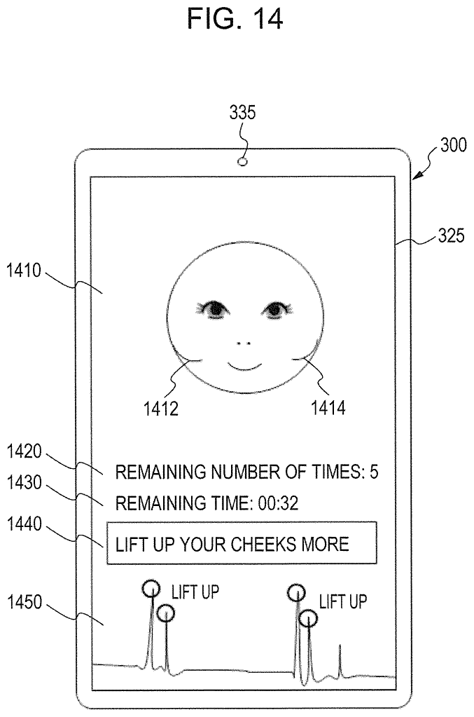

FIG. 14 is an explanatory diagram illustrating an example process according to the exemplary embodiment.

The smartphone 300 includes a screen 325 and the camera 335. The camera 335 is also referred to as a built-in camera and is used to capture an image of the face of the user 390 having the smartphone 300. FIG. 14 illustrates an example in which an image of the face of the user 390 is captured by the camera 335 and information detected by the wearable device 350 is displayed in the form of a graph.

The example in FIG. 14 is used for a facial exercise for beauty, training of facial muscles of expression, or the like. In this example, a face image of the user 390 and a measurement result of biological information are displayed together.

The screen 325 includes a captured image display area 1410, a remaining number of times display area 1420, a remaining time display area 1430, a comment display area 1440, and a graph display area 1450.

At the time of initial setting of the wearable device 350, initialization is performed in accordance with a potential of an individual user. At the time of initialization, both or either of a potential and image data is used.

In a case where it is not possible to measure a potential, the number of times of training is counted from only image information. The training may be, for example, training for orbicular muscles of the eyes. The “case where it is not possible to measure a potential” is an example of a case where it is not possible to extract biological information.

The screen 325 of the smartphone 300 is displayed by a training application, and displays the face of the user 390, potential data, and activity information in association with each other. The activity information includes, for example, an advice to open the eyes wider, the number of times of training, a training time, and the like. For example, “remaining number of times: 5” is displayed in the remaining number of times display area 1420, “remaining time: 00:32” is displayed in the remaining time display area 1430, and “lift up your cheeks more” is displayed in the comment display area 1440.

In addition, myoelectric potential data received from the wearable device 350 is analyzed to detect whether lift-up or the like has been performed. Furthermore, a face image may be analyzed to detect whether lift-up or the like has been performed. That is, even in an abnormal state where it is not possible to obtain information from a biological potential such as a myoelectric potential, an effect of the training may be estimated and the user 390 may be notified of the effect without the application being stopped. Similarly, in a case where it is not possible to obtain a face image, an effect may be estimated from only information about a biological potential and the user 390 may be notified of the effect. In a case where both the biological potential and the face image are normally measured and analyzed, a training effect can be estimated and notified more accurately than in a case where only one of them is measured.

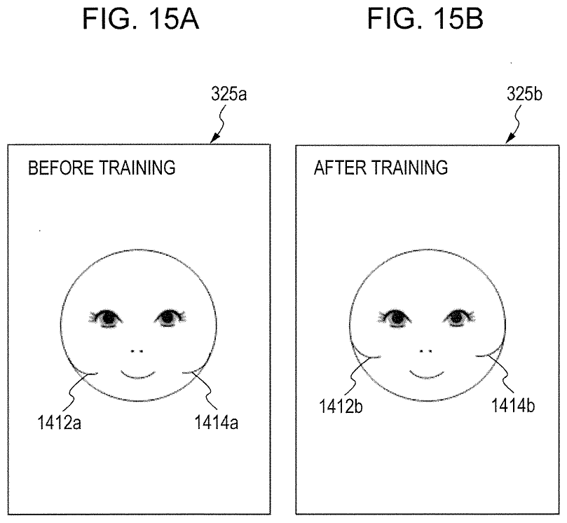

FIGS. 15A and 15B are explanatory diagrams illustrating an example process according to the exemplary embodiment.

In this example, face images before and after training are displayed in a comparable manner. A screen 325a illustrated in FIG. 15A displays a face image before training, and a screen 325b illustrated in FIG. 15B displays a face image after training. In this example, it is seen that a cheek line 1412a and a cheek line 1414a are raised and changed to a cheek line 1412b and a cheek line 1414b by the training.

For comparison of the images, a manner of displaying the images may be selected from among, for example, displaying the images side by side within one screen, displaying the images one by one, making two images transparent and superimposing the images one on top of the other, displaying right or left halves of the images before and after training, and enlarging part of the images.

In addition, a face image captured when lift-up or the like is performed may be displayed. Furthermore, the face images before and after the lift-up may also be displayed.

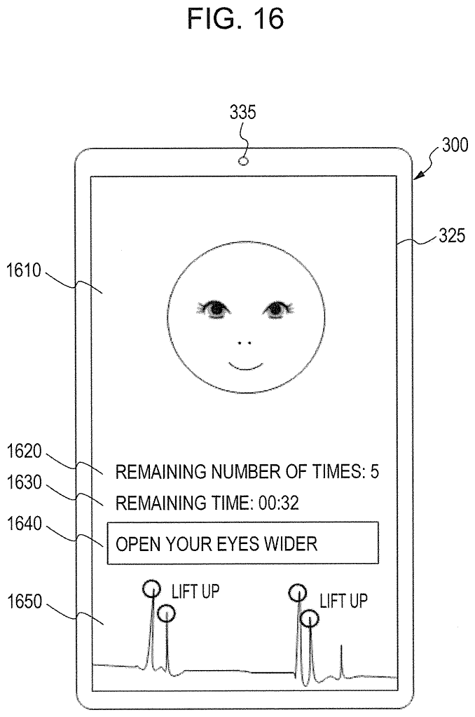

FIG. 16 is an explanatory diagram illustrating an example process according to the exemplary embodiment. The example illustrated in FIG. 16 is used for a facial exercise for beauty, training of facial muscles of expression, or the like. In this example, a face image of the user 390 and a measurement result of biological information are displayed together.

The screen 325 includes a captured image display area 1610, a remaining number of times display area 1620, a remaining time display area 1630, a comment display area 1640, and a graph display area 1650.

The example illustrated in FIG. 16 is similar to the example illustrated in FIG. 14, but is about training for orbicular muscles of the eyes.

For example, “remaining number of times: 5” is displayed in the remaining number of times display area 1620, “remaining time: 00:32” is displayed in the remaining time display area 1630, and “open your eyes wider” is displayed in the comment display area 1640.

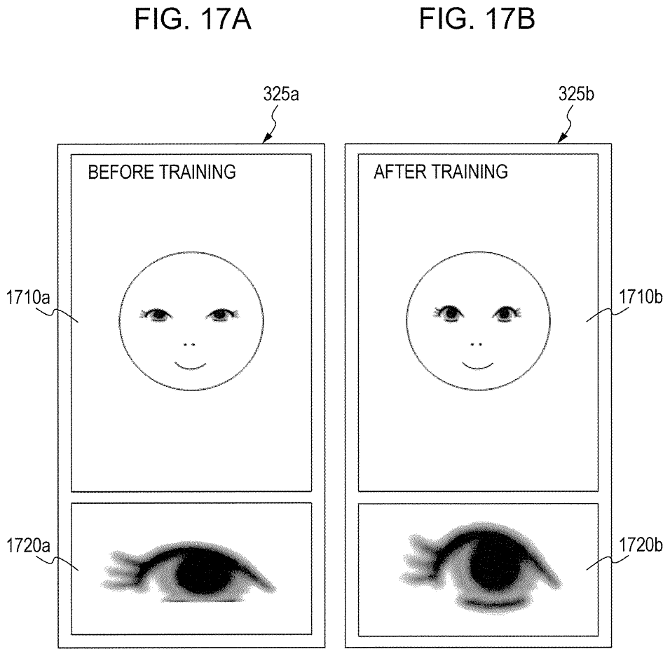

FIGS. 17A and 17B are explanatory diagrams illustrating an example process according to the exemplary embodiment.

In this example, face images before and after training are displayed in a comparable manner. The screen 325a illustrated in FIG. 17A displays a face image before training, and includes a captured image display area 1710a and a training target display area 1720a. The screen 325b illustrated in FIG. 17B displays a face image after training, and includes a captured image display area 1710b and a training target display area 1720b.

In this example, it is seen that the degree of opening of the eyes is increased. In particular, the target portion of training is enlarged. In this example, the image of an eye in the training target display area 1720a and the image of an eye in the training target display area 1720b can be compared with each other.

For comparison of the images, a manner of displaying the images may be selected from among, for example, displaying the images side by side within one screen, displaying the images one by one, making two images transparent and superimposing the images one on top of the other, displaying right or left halves of the images before and after training, and enlarging part of the images.

In addition, a face image captured when opening of the eyes or the like is performed may be displayed. Furthermore, the face images before and after the opening of the eyes may also be displayed.

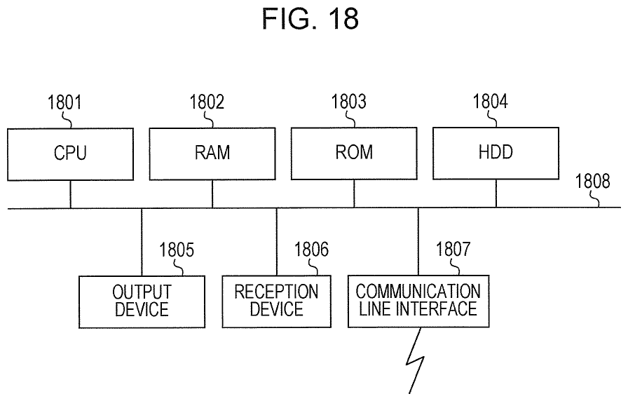

A hardware configuration of a computer that executes a program as the exemplary embodiment is a typical computer as illustrated in FIG. 18 and is specifically a personal computer, a computer that can be a server, or the like. Specifically, a central processing unit (CPU) 1801 is used as a processing unit (computing unit), and a random access memory (RAM) 1802, a read only memory (ROM) 1803, and a hard disk drive (HDD) 1804 are used as a storage device. As the HDD 1804, an HDD, a solid state drive (SSD), which is a flash memory, or the like may be used, for example. The hardware configuration of the computer includes the CPU 1801 that executes a program of the biological information extracting module 105, the head information extracting module 110, the analyzing module 115, the display control module 120, the display module 125, the communication module 230, the communication module 255, the biological information detecting module 260, and the like; the RAM 1802 storing the program and data; the ROM 1803 storing a program or the like for activating the computer; the HDD 1804 serving as an auxiliary storage device that stores brain wave information, head information, and the like; a reception device 1806 that receives data in accordance with a user operation (including a motion, sound, line of sight, and the like) performed on a keyboard, mouse, touch screen, microphone, camera (including a line-of-sight detecting camera or the like), or the like; an output device 1805, such as a cathode ray tube (CRT), a liquid crystal display, or a speaker; a communication line interface 1807 for connecting to a communication network, such as a network interface card; and a bus 1808 that connects these devices to transmit and receive data. Plural computers each having the above-described hardware configuration may be connected to each other through a network.

In the above-described exemplary embodiment, the process based on a computer program is performed by cooperation between software and hardware resources by causing a system having the above-described hardware configuration to read the computer program as software. Accordingly, the above-described embodiment is carried out.

The hardware configuration illustrated in FIG. 18 is one example configuration. The exemplary embodiment is not limited to the configuration illustrated in FIG. 18 and may adopt any configuration capable of executing the modules described in the exemplary embodiment. For example, one or some of the modules may be constituted by dedicated hardware (for example, an application specific integrated circuit (ASIC), a reconfigurable integrated circuit (a field-programmable gate array (FPGA)), or the like), or one or some of the modules may be included in an external system and connected through a communication line. Furthermore, plural systems each having the hardware configuration illustrated in FIG. 18 may be connected to each other through a communication line and may operate in cooperation with each other. In particular, one or some of the modules may be incorporated in a mobile information communication device (including a mobile phone, a smartphone, a mobile device, a wearable computer, and the like), a home information appliance, a robot, or the like.

The above-described program may be provided by storing it in a recording medium or may be provided through communication. In this case, for example, the above-described program may be regarded as a “computer-readable recording medium storing the program”.

The “computer-readable recording medium storing the program” is a computer-readable recording medium storing the program and used to install, execute, or distribute the program.

Examples of the recording medium include a digital versatile disc (DVD), such as “DVD-R, DVD-RW, DVD-RAM, and the like” defined by DVD Forum and “DVD+R, DVD+RW, and the like” defined by DVD+RW Alliance; a compact disc (CD), such as a read only memory (CD-ROM), a CD recordable (CD-R), and a CD rewritable (CD-RW); a Blu-ray Disc (registered trademark); a magneto-optical (MO) disc; a flexible disk (FD); magnetic tape; a hard disk; a read only memory (ROM); an electrically erasable and programmable ROM (EEPROM, registered trademark); a flash memory; a random access memory (RAM); and a secure digital (SD) memory card.

All or part of the above-described program may be stored or distributed by recording it on the recording medium. Alternatively, all or part of the program may be transmitted through communication, for example, using a transmission medium such as a wired or wireless communication network used in a local area network (LAN), a metropolitan area network (MAN), a wide area network (WAN), the Internet, an intranet, or an extranet, or a combination of the wired and wireless communication networks. Alternatively, all or part of the program may be carried using carrier waves.

Furthermore, the above-described program may be all or part of another program, or may be recorded on a recording medium together with another program. Alternatively, the program may be recorded on plural recording media in a split manner. The program may be recorded in any manner, for example, the program may be compressed or encrypted, as long as the program can be recovered.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing device comprising:
an extractor that extracts biological information and a motion of a head from a potential measurement result, which is a result of measuring a potential in the head of a human body; and
a controller that performs control to enable the biological information and the motion of the head that have been extracted by the extractor to be simultaneously presented in association with each other.

2. The information processing device according to claim 1, wherein
the extractor extracts waves in a plurality of frequency bands from the potential measurement result, and
the controller performs control to display, on the screen in a comparable manner, the waves and the motion of the head that have been extracted by the extractor.

3. The information processing device according to claim 1, wherein
the motion of the head includes chewing,
the extractor extracts, as a chewing portion, a portion that matches a predetermined pattern in the potential measurement result, and
the controller performs control to display in a comparable manner the biological information and the chewing portion that is on a graph indicating the potential measurement result.

4. The information processing device according to claim 3, wherein the predetermined pattern is that a first peak of the graph is higher than a predetermined first threshold value or is higher than or equal to the first threshold value and that a second peak after the first peak is lower than the first threshold value or is lower than or equal to the first threshold value and is higher than a predetermined second threshold value or is higher than or equal to the second threshold value, the second threshold value being smaller than the first threshold value.

5. The information processing device according to claim 1, wherein
the motion of the head includes swallowing,
the information processing device further comprises an analyzer that analyzes that a portion that matches a predetermined pattern in the potential measurement result is a swallowing portion, and
the controller performs control to display in a comparable manner the biological information and the swallowing portion that is on a graph indicating the potential measurement result.

6. The information processing device according to claim 5, wherein the predetermined pattern is that a third peak of the graph after chewing is higher than a predetermined third threshold value or is higher than or equal to the third threshold value.

7. The information processing device according to claim 6, wherein if the analyzer analyzes that the motion of the head is a predetermined motion by using a measurement result of an acceleration sensor put on the head, the analyzer extracts the swallowing as swallowing of drink.

8. The information processing device according to claim 7, wherein the predetermined motion is a motion of tilting the head backward.

9. The information processing device according to claim 1, wherein the controller performs control to simultaneously display results of a plurality of types of transform processes on the potential measurement result.

10. The information processing device according to claim 1, wherein the controller performs control to display results of spectrum analysis for a predetermined period using a low-pass filter and a high-pass filter and to display other data that does not have periodicity.

11. The information processing device according to claim 1, further comprising an image capturing unit that captures an image of the head of the human body, wherein
the controller further performs control to display the image captured by the image capturing unit.

12. The information processing device according to claim 11, wherein a state of a user is presented, with the captured image and the biological information being associated with each other.

13. The information processing device according to claim 12, wherein if the biological information is not extracted, the state of the user is presented by analyzing the image.

14. A non-transitory computer readable medium storing a program causing a computer to execute a process for information processing, the process comprising:
extracting biological information and a motion of a head from a potential measurement result, which is a result of measuring a potential in the head of a human body; and
performing control to enable the biological information and the motion of the head that have been extracted to be simultaneously presented in association with each other.

15. An information processing device comprising:
extraction means for extracting biological information and a motion of a head from a potential measurement result, which is a result of measuring a potential in the head of a human body; and
control means for performing control to enable the biological information and the motion of the head that have been extracted by the extraction means to be simultaneously presented in association with each other.
